(19) United States
(12) Patent Application Publication
(10) Pub. No.: US 2024/0133863 A1
(43) Pub. Date: Apr. 25, 2024

(54) LIPID BILAYER SUBSTRATE AND PRODUCTION METHOD THEREOF

(71) Applicant: NIPPON TELEGRAPH AND TELEPHONE CORPORATION, Tokyo (JP)

(72) Inventors: Yoshiaki KASHIMURA, Musashino-shi, Tokyo (JP); Masumi YAMAGUCHI, Musashino-shi, Tokyo (JP)

(73) Assignee: NIPPON TELEGRAPH AND TELEPHONE CORPORATION, Tokyo (JP)

(21) Appl. No.: 18/276,618

(22) PCT Filed: Feb. 25, 2021
(86) PCT No.: PCT/JP2021/007078

§ 371 Date: Aug. 9, 2023

(51) Int. Cl. G01N 33/487

(52) U.S. Cl. CPC G01N 33/48728

(57) ABSTRACT

A lipid bimolecular membrane substrate 1A includes: a substrate 12 in which a microwell 20 opened on one surface is formed; a lipid bimolecular membrane 30 disposed on the substrate 12 so as to cover an opening of the microwell 20; and a sealing liquid 22 disposed between the substrate 12 and the lipid bimolecular membrane 30. The sealing liquid 22 contains an ionic liquid.

### TECHNICAL FIELD

The present invention relates to a lipid bimolecular membrane substrate and a method for manufacturing the lipid bimolecular membrane substrate.

### BACKGROUND ART

In molecular biological basic studies, completion of human genome planning for the purpose of decoding the entire base sequence of human DNA was declared in 2003, and now it is entering a post-genomic era of examining information encoded in a genome.

In the post-genomic era, a function of a protein has been actively studied. Among proteins, a membrane protein is responsible for transport of substances necessary for life support and signal transduction, and is deeply involved in various diseases and physiological functions such as a drug response and an immune reaction. Therefore, attempts have been made to control the structure of a membrane protein present in a cell membrane and to utilize and elucidate a function of the membrane protein.

As a representative method for disposing a membrane protein in vitro, a method is known in which a lipid molecule is dissolved in an organic solvent such as n-decane, the lipid solution is applied to a small pore formed on a substrate in an aqueous solution to form a black membrane, and a membrane protein is fused to the black membrane (see, for example, Non Patent Literature 1).

In the method for fusing a membrane protein to a black membrane, it has been pointed out that a residual organic solvent and non-uniformity in the black membrane may affect the physiological activity of the membrane protein. In addition, the black membrane has low stability, and the black membrane has a lifetime of at most about several hours.

Meanwhile, the present inventors have formed a micrometer-scale microwell on a semiconductor substrate and have developed a giant lipid membrane vesicle on an electrolyte solution dropped into the microwell, thereby preparing a lipid bimolecular membrane substrate in which an opening of the microwell is covered with a lipid membrane. Furthermore, the inventors have succeeded in reconstituting a membrane protein in a lipid bimolecular membrane covering a microwell (Non Patent Literature 2).

Since the above-described lipid bimolecular membrane substrate is prepared using a giant lipid membrane vesicle, the lipid bimolecular membrane covering the microwell does not contain an organic solvent. In addition, the lipid bimolecular membrane has a lifetime of about several days, which is much longer than the lifetime of the above-described black membrane. In addition, it has been confirmed by an optical technique (fluorescence observation) that a membrane protein reconstituted in a lipid bimolecular membrane covering a microwell has activity.

As described above, a substrate in which a lipid bimolecular membrane containing a membrane protein is reconstituted is useful as a platform for measuring a function of the membrane protein.

### CITATION LIST

### Non Patent Literature

Non Patent Literature 1: “New Patch Clamp Experimental Technique”, edited by Yasunobu Okada (2001, Yoshioka Shoten)
Non Patent Literature 2: K. Sumitomo et al. Ca2+ ion transport through channels formed by α-hemolysin analyzed using a microwell array on a Si substrate, Biosensors and Bioelectronics, 2012: 31; 445-450.

### SUMMARY OF INVENTION

### Technical Problem

However, a result suggesting that inorganic ions such as metal ions and halogen ions move (hereinafter, this may be referred to as ion leakage) from the outside of the microwell to the inside thereof or from the inside of the microwell to the outside thereof even in a state where no membrane protein is present on the lipid bimolecular membrane was observed.

When characteristics of an ion channel are analyzed using a lipid bimolecular membrane holding a membrane protein such as an ion channel, such ion leakage may be noise and affect a measurement result.

Therefore, an object of the present invention is to provide a lipid bimolecular membrane substrate suppressing ion leakage and a method for manufacturing the lipid bimolecular membrane substrate.

### Solution to Problem

An aspect of the present invention is a lipid bimolecular membrane substrate including: a substrate in which a microwell opened on one surface is formed; a lipid bimolecular membrane disposed on the substrate so as to cover an opening of the microwell; and a sealing liquid disposed between the substrate and the lipid bimolecular membrane, in which the sealing liquid contains an ionic liquid.

Another aspect of the present invention is a method for manufacturing a lipid bimolecular membrane substrate, the method including: a step of dropping a sealing liquid onto a substrate in which a microwell opened on one surface is formed, filling the inside of the microwell with the sealing liquid, and covering a peripheral edge of the microwell with the sealing liquid; a step of adding a lipid membrane vesicle to the sealing liquid on the microwell; and a step of developing the lipid membrane vesicle in the sealing liquid to cover an opening of the microwell with a lipid bimolecular membrane, in which the sealing liquid contains an ionic liquid.

### Advantageous Effects of Invention

The present invention can provide a lipid bimolecular membrane substrate suppressing ion leakage and a method for manufacturing the lipid bimolecular membrane substrate.

### BRIEF DESCRIPTION OF DRAWINGS

FIG. 1 is a perspective view of an embodiment of a lipid bimolecular membrane substrate according to the present invention.

FIG. 2 is a cross-sectional view of an embodiment of the lipid bimolecular membrane substrate according to the present invention.

FIG. 3 is a diagram schematically illustrating ion leakage in a conventional lipid bimolecular membrane substrate.

FIG. 4 is a cross-sectional view of an embodiment of the lipid bimolecular membrane substrate according to the present invention.

FIG. 5A is a schematic cross-sectional view of a laminate.

FIG. 5B is a schematic cross-sectional view of a laminate in which a recess is formed.

FIG. 5C is a schematic cross-sectional view of a substrate in which a microwell is formed.

FIG. 5D is a schematic cross-sectional view of a substrate to which a sealing liquid has been dropped.

FIG. 5E is a cross-sectional view schematically illustrating a state in which a lipid membrane vesicle is developed to cover an opening of a microwell.

FIG. 6A is a schematic cross-sectional view of a second layer.

FIG. 6B is a schematic cross-sectional view of the second layer on which an electrode is laminated.

FIG. 6C is a schematic cross-sectional view of a laminate in which a first layer and a resist film are laminated on the second layer.

FIG. 6D is a schematic cross-sectional view of the laminate after the resist film is etched.

FIG. 6E is a schematic cross-sectional view of the laminate in which a through-hole is formed in the first layer.

FIG. 6F is a schematic cross-sectional view of the laminate after the through-hole is etched.

FIG. 6G is a schematic cross-sectional view of the laminate after the resist film is removed.

FIG. 7 is a photograph of a state in which a fluorescent molecule is put in a microwell and confined by a lipid bimolecular membrane, taken with a fluorescence microscope.

FIG. 8 is a photograph of a state in which calcium flows into a microwell, taken with a fluorescence microscope.

FIG. 9 is a photograph of a plating layer laminated on an electrode on a bottom surface of a microwell, taken with an electron microscope.

### DESCRIPTION OF EMBODIMENTS

In a conventional method for manufacturing a lipid bimolecular membrane substrate, first, an electrolyte solution containing potassium ions and the like is dropped onto a semiconductor substrate in which a microwell is formed. Subsequently, a giant lipid membrane vesicle is developed on the electrolyte solution dropped into the microwell to obtain a lipid bimolecular membrane substrate in which an opening of the microwell is covered with a lipid bimolecular membrane.

It is known that a thin interfacial aqueous layer having a thickness of about 1 to 2 nm is present between a substrate and a lipid bimolecular membrane (for example, Johonson S J et al., Structure of an adsorbed dimyristoylphosphatidylcholine bilayer measured with specular reflection of neutrons, Biophys J. 1991; 59: 289-94).

It is considered that ion diffusion through the interfacial aqueous layer is a cause of movement of inorganic ions such as metal ions and halide ions (that is, ion leakage) from the outside of the microwell to the inside thereof or from the inside of the microwell to the outside thereof (for example, Forbes et al., Hermetically sealed microwell with a lipid bilayer created using a self-assembled monolayer, Appl. Phys. Express. 2015; 8: 117201).

The present inventors have come to an idea that ion leakage through the interfacial aqueous layer can be suppressed by sealing a gap between the substrate and the lipid bimolecular membrane with an ionic liquid aqueous solution containing an ionic liquid without sealing the gap with an inorganic electrolyte solution such as potassium chloride or sodium chloride.

[Lipid Bimolecular Membrane Substrate]

### First Embodiment

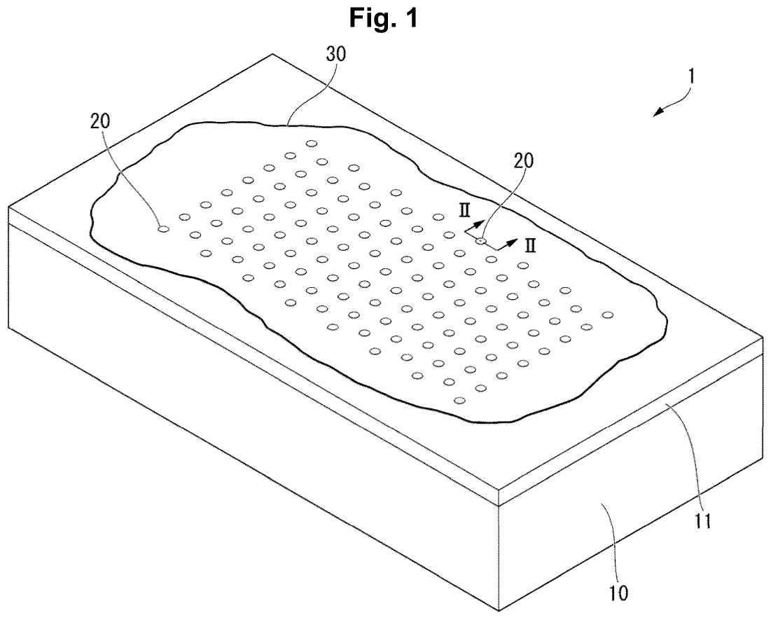

A lipid bimolecular membrane substrate according to an embodiment will be described with reference to the drawings. FIG. 1 is a perspective view of a lipid bimolecular membrane substrate 1.

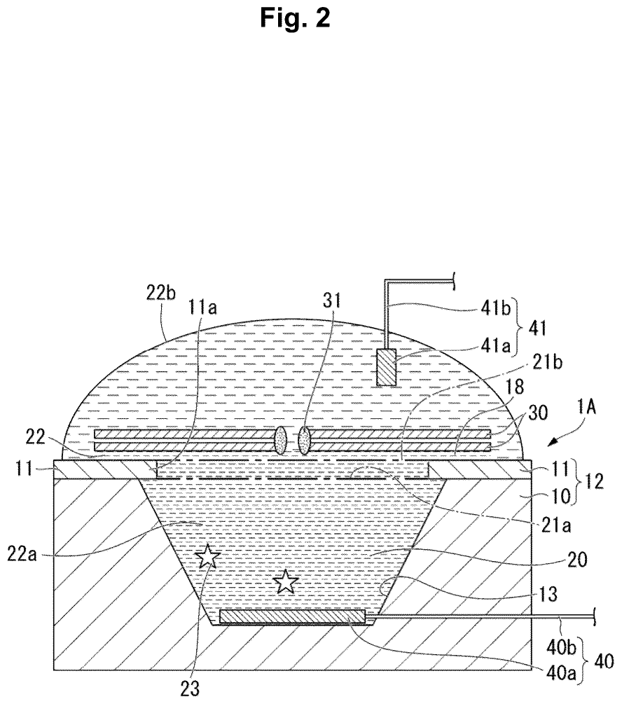

FIG. 2 is a cross-sectional view taken along line II-II of the lipid bimolecular membrane substrate 1 of FIG. 1.

As illustrated in FIGS. 1 and 2, a lipid bimolecular membrane substrate 1A includes a substrate 12 and a lipid bimolecular membrane 30 disposed on the substrate 12. A microwell 20 is formed in the substrate 12. The lipid bimolecular membrane 30 is disposed so as to cover an opening 21b of the microwell 20.

The substrate 12 includes a substrate body 10 in which a recess 13 opened on one surface is formed, and a thin film layer 11 laminated on the substrate body 10.

A material of the substrate body 10 is not particularly limited, and examples thereof include silicon, a silicon oxide, a silicon nitride, quartz, mica, glass, and plastic. As the material of the substrate body 10, it is preferable to use a material in which a surface of the substrate body 10 is negatively charged in an aqueous solution having a pH of 3 to 10.

The thickness and shape of the substrate body 10 are appropriately adjusted according to an application.

As a method for forming the recess 13 on the substrate body 10, a known method can be applied. For example, a microfabrication technique such as a method combining a photolithography method and a dry etching method or an electron beam lithography method can be applied.

The thin film layer 11 has an overhang portion 11a protruding in a direction of narrowing an opening 21a of the recess 13. The opening 21b of the thin film layer 11 is smaller than the opening 21a of the recess 13.

As a material of the thin film layer 11, a known material is applied, and the material may be the same as the material of the substrate body 10, but is preferably different from the material of the substrate body 10. This facilitates etching when the overhang portion 11a is formed.

When a silicon oxide or a silicon nitride is used as the material of the thin film layer 11, the thin film layer 11 constituting a surface of the substrate body 10 has a negative surface charge in a neutral solution.

A method for forming the thin film layer 11 may be, for example, a method for sputtering the material of the thin film layer 11 on the substrate body 10.

Alternatively, the method for forming the thin film layer 11 may be a method for forming the thin film layer 11 of a silicon oxide film on a silicon substrate by a thermal oxidation method when the substrate body 10 is a silicon substrate.

The microwell 20 is a space surrounded by the recess 13, the overhang portion 11a, and the opening 21b of the thin film layer 11.

The thin film layer 11 does not have to have the overhang portion 11a. In this case, the opening 21a of the recess 13 has the same size as the opening 21b of the thin film layer 11. In this case, the microwell 20 is a space surrounded by the recess 13 and the opening 21b of the thin film layer 11.

The substrate 12 does not have to have the thin film layer 11. In this case, the microwell 20 is a space surrounded by the recess 13 and the opening 21a of the recess 13.

The number of the microwells 20 included in the substrate 12 is not particularly limited, and may be one or more, and for example, may be 1 to 10000.

The opening 21a of the recess 13 and the opening 21b of the microwell 20 preferably each have a diameter of 100 nm or more. As a result, a membrane protein 31 having a size of 10 to 20 nm can be disposed on the lipid bimolecular membrane 30.

The opening 21a of the recess 13 and the opening 21b of the microwell 20 preferably each have a diameter of 5 μm or less. As a result, a lipid membrane vesicle 60 can be developed to cover the opening 21b.

The depth of the microwell 20 only needs to be appropriately designed according to the diameters of the opening 21a and the opening 21b, and is, for example, a depth of 0.1 μm or more and 10 μm or less.

The lipid bimolecular membrane 30 may contain a three-component system of a saturated lipid, an unsaturated lipid, and cholesterol.

The type of lipid molecule of the lipid bimolecular membrane 30 is not particularly limited as long as the lipid molecule can form a lipid bimolecular membrane (lipid bilayer membrane), and examples thereof include: a neutral or anionic lipid molecule such as phosphatidylcholine (PC), phosphatidylethanolamine (PE), phosphatidylserine (PS), phosphatidylinositol (PI), phosphatidylinositol phosphate (PIP), phosphatidic acid (PA), phosphatidylglycerol (PG), or sphingolipid; and a cationic lipid molecule such as 1,2-dioleyl-3-trimethylammoniumpropane, trimethylammoniumpropane (TAP), or ethylphosphocholine (EPC). These lipid molecules may be used singly or in combination of two or more types thereof.

A method for forming the lipid bimolecular membrane 30 will be described in detail later, and examples thereof include a method for developing a lipid membrane vesicle on the substrate 12. Here, the “lipid membrane vesicle” is a vesicle (small sac) formed by a lipid bimolecular membrane (lipid bilayer membrane).

In the present specification, “a lipid bimolecular membrane is developed” means that a lipid membrane vesicle is cleaved to form a lipid bilayer membrane. The lipid bilayer membrane covers the opening 21b of the microwell 20.

As a material of the substrate body 10 or the thin film layer 11, a material that is negatively charged in an aqueous solution having a pH of 3 to 10 is preferably used. As a result, the lipid bilayer membrane is attracted to a surface of the negatively charged thin film layer 11 by electrostatic attraction, and the opening 21b of the microwell 20 is easily covered with the lipid bimolecular membrane 30.

The membrane protein 31 is introduced into the lipid bimolecular membrane 30.

Examples of a method for introducing the membrane protein 31 into the lipid bimolecular membrane 30 include a method in which a proteoliposome into which the target membrane protein 31 is previously introduced is prepared by a known method, and the proteoliposome is fused to the lipid bimolecular membrane 30. As a result, the target membrane protein 31 is introduced into the lipid bimolecular membrane 30.

In the lipid bimolecular membrane substrate 1A, a gap (gap 18) is present between the substrate 12 and the lipid bimolecular membrane 30. The gap 18 has a width of 1 to 2 nm in a thickness direction, and the gap 18 is sealed with a sealing liquid 22. The sealing liquid 22 contains an ionic liquid. Details of the ionic liquid contained in the sealing liquid 22 will be described later.

The inside of the microwell 20 is filled with an internal liquid 22a. Although details will be described later, the composition of the internal liquid 22a is the same as the composition of the sealing liquid 22.

The internal liquid 22a contains a fluorescent molecule (fluorescent probe) 23. When ions and the like are exchanged between an external liquid 22b covering the lipid bimolecular membrane 30 and the internal liquid 22a via the membrane protein 31, the exchange of ions can be observed by observing a fluorescence intensity of the fluorescent molecule 23 with a fluorescence microscope.

The internal liquid 22a does not have to contain the fluorescent molecule 23. In this case, as described later, an electrode unit 40 and a counter electrode unit 41 are connected to a measuring device, and a current (ion flow) or the like passing through pores of the membrane protein 31 of the lipid bimolecular membrane 30 is measured, whereby a function of the membrane protein 31 introduced into the lipid bimolecular membrane 30 can be analyzed electrophysiologically.

Examples of the fluorescent molecule (fluorescent probe) 23 include a water-soluble molecule that emits fluorescence when a state change occurs inside the microwell 20. By measuring a fluorescence intensity change of the fluorescent molecule 23, it is possible to analyze a function of the membrane protein introduced into the lipid bimolecular membrane 30.

Specific examples of the fluorescent molecule 23 include Fluo4 and Quin2 whose fluorescence intensities change with a change in calcium ion concentration inside the microwell 20. In a case where a membrane protein having calcium ion permeability is introduced into the lipid bimolecular membrane 30, when calcium ions pass through the lipid bimolecular membrane 30 via the membrane protein, and the concentration of calcium ions in the microwell 20 changes, a fluorescence intensity from the fluorescent molecule changes. By detecting this change with a fluorescence microscope or the like, the function of the membrane protein can be analyzed.

As illustrated in FIG. 2, an upper portion of the lipid bimolecular membrane 30 and a peripheral edge of the opening 21b of the microwell 20 are covered with the external liquid 22b.

The type of a solute of the external liquid 22b is not particularly limited. Examples of the solute include a pH buffer (for example, HEPES, MOPS, TRIS, or a phosphate) for adjusting the pH of each solution; a salt (for example, NaCl, KCl, CaCl₂, MgCl₂, or a physiological salt); a saccharide (glucose, sucrose, or the like); an alcohol (for example, glycerol or xylitol); and a glycol (for example, ethylene glycol, propylene glycol, or polyethylene glycol).

In the lipid bimolecular membrane substrate 1A, a gap (gap 18) is present between the substrate 12 and the lipid bimolecular membrane 30. The gap 18 has a width of 1 to 2 nm in a thickness direction, and the gap 18 is sealed with a sealing liquid 22. The sealing liquid 22 contains an ionic liquid.

The ionic liquid refers to a salt that exists as a liquid at normal temperature. The ionic liquid is a salt that contains a cation and an anion, has a weak electrostatic interaction between these ions, and is hardly crystallized. In the present specification, the normal temperature is 10 to 40° C.

The ionic liquid has a melting point of 100° C. or lower, and further has the following characteristics 1) to 5). Characteristic 1) having an extremely low vapor pressure Characteristic 2) exhibiting non-flammability in a wide temperature range Characteristic 3) maintaining a liquid state in a wide temperature range Characteristic 4) being able to change a density Characteristic 5) being able to control a polarity

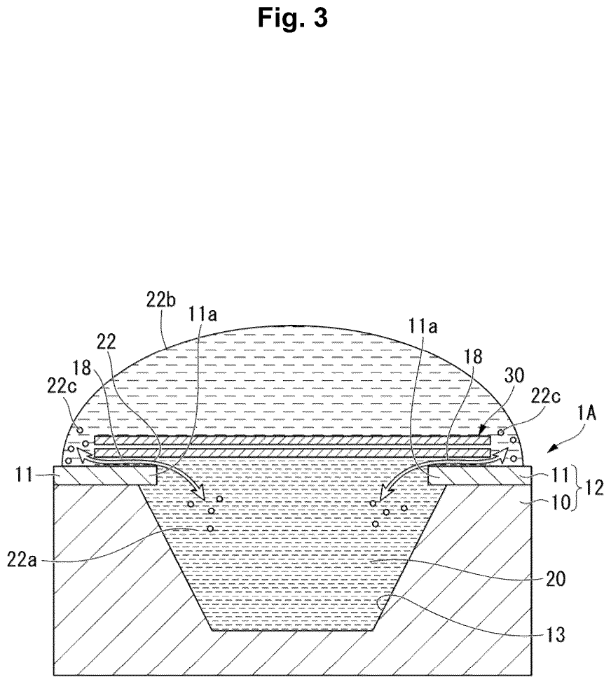

FIG. 3 is an example of a conventional lipid bimolecular membrane substrate. The sealing liquid 22 and the internal liquid 22a of the lipid bimolecular membrane substrate contain an inorganic ion 22c.

Here, the inorganic ion means an inorganic cation and an inorganic anion.

Examples of the inorganic cation include a metal ion. Examples of the metal ion include a lithium ion, a sodium ion, a magnesium ion, an aluminum ion, a potassium ion, a calcium ion, a manganese ion, an iron ion, a cobalt ion, a nickel ion, a copper ion, and a zinc ion.

Examples of the inorganic anion include a halide ion. Examples of the halide ion include a fluoride ion, a chloride ion, a bromide ion, and an iodide ion.

The gap 18 has a width of 1 to 2 nm in a thickness direction. Meanwhile, the inorganic ion has an ionic radius of 0.05 to 0.2 nm.

When the sealing liquid 22 and the internal liquid 22a contain the inorganic ion 22c, the inorganic ion 22c can pass through the gap 18. Therefore, the inorganic ion 22c can move between the internal liquid 22a of the microwell 20 and the external liquid 22b thereof.

Meanwhile, in the lipid bimolecular membrane substrate 1A according to an embodiment of the present invention, a cation and an anion contained in the ionic liquid are larger than the width of the gap 18 in the thickness direction. Therefore, the cation or the anion cannot pass through the gap 18 at all or cannot substantially pass through the gap 18.

Here, in the conventional lipid bimolecular membrane substrate illustrated in FIG. 3, a case where the external liquid 22b contains a calcium ion, and the sealing liquid 22 in the gap 18 and the internal liquid 22a contain a potassium ion will be described.

When a calcium ion contained in the external liquid 22b passes through the gap 18 to flow into the internal liquid 22a, the internal liquid 22a needs to be electrically neutral. Therefore, a potassium ion contained in the internal liquid 22a passes through the gap 18 to flow out to the external liquid 22b.

Meanwhile, a case where the external liquid 22b contains a calcium ion and the sealing liquid 22 and the internal liquid 22a contain an ionic liquid in the lipid bimolecular membrane substrate 1A according to an embodiment of the present invention illustrated in FIG. 2 will be described.

In this case, a cation of the ionic liquid contained in the internal liquid 22a cannot pass through the gap 18 to flow out to the external liquid 22b. Therefore, it is presumed that a calcium ion contained in the external liquid 22b cannot pass through the gap 18 to flow into the internal liquid 22a.

As described above, it is presumed that since the sealing liquid 22 in the gap 18 and the internal liquid 22a contain the ionic liquid, an inorganic cation contained in the external liquid 22b can be suppressed from passing through the gap 18 to flow into the internal liquid 22a.

Similarly, it is presumed that since the sealing liquid 22 in the gap 18 and the internal liquid 22a contain the ionic liquid, an inorganic anion contained in the external liquid 22b can be suppressed from passing through the gap 18 to flow into the internal liquid 22a.

When it is desired to measure the amount of ions passing through pores of the membrane protein 31 of the lipid bimolecular membrane 30, the amount of ions passing through the gap 18 is reflected as noise with respect to a measured value.

Since the sealing liquid 22 in the gap 18 and the internal liquid 22a contain the ionic liquid, the amount of ions passing through the gap 18 is reduced, and noise is reduced. Therefore, it is possible to more accurately measure the amount of ions passing through pores of the membrane protein 31 of the lipid bimolecular membrane 30.

The ionic liquid contained in the sealing liquid 22 and the internal liquid 22a contains one or more types of cations. In addition, the ionic liquid contained in the sealing liquid 22 and the internal liquid 22a contains one or more types of anions.

Examples of the cation contained in the ionic liquid include cations represented by the following formulas (ca-1) to (ca-5).

In the present specification, the simple term “cation” does not include the above-described inorganic cation.

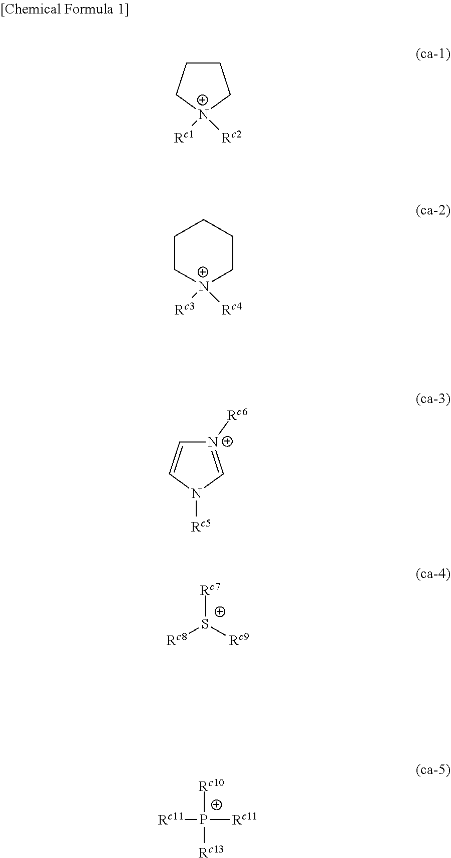

in which R^{c1} to R^{c6} each independently represent an alkyl group having 1 to 12 carbon atoms. R^{c7} to R^{c9} each independently represent an aryl group optionally having a substituent. R^{c10} to R^{c13} each independently represent a linear alkyl group having 1 to 20 carbon atoms.

The cation contained in the ionic liquid preferably has an ionic radius larger than the gap 18 between the lipid bimolecular membrane 30 and the substrate 12. More specifically, the ionic radius of the cation contained in the ionic liquid is preferably 1 nm or more, more preferably 1.5 nm or more, and still more preferably 2 nm or more.

Examples of the anion contained in the ionic liquid include cations represented by the following formulas (an-1) to (an-3).

In the present specification, the simple term “anion” does not include the above-described inorganic anion.

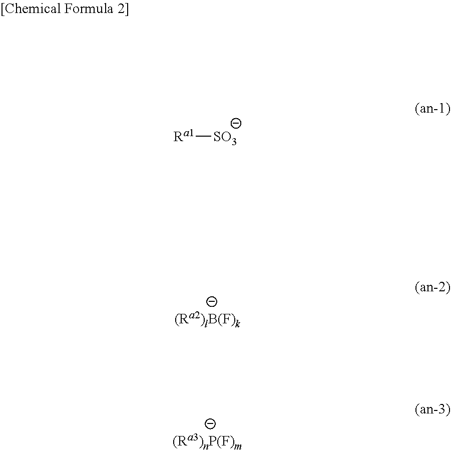

In formula (an-1), Rat represents an aromatic hydrocarbon group optionally having a substituent, an aliphatic cyclic group optionally having a substituent, or a chain hydrocarbon group optionally having a substituent. In formula (an-2), R^{a2} represents an alkyl group having 1 to 5 carbon atoms and optionally having a substituent of a fluorine atom. k represents an integer of 1 to 4, 1 represents an integer of 0 to 3, and k+1=4 is satisfied. In formula (an-3), R^{a3} represents an alkyl group having 1 to 5 carbon atoms and optionally having a substituent of a fluorine atom. m represents an integer of 1 to 6, n represents an integer of 0 to 5, and m+n=6 is satisfied.

The anion contained in the ionic liquid preferably has an ionic radius larger than the gap 18 between the lipid bimolecular membrane 30 and the substrate 12. More specifically, the ionic radius of the anion contained in the ionic liquid is preferably 1 nm or more, more preferably 1.5 nm or more, and still more preferably 2 nm or more.

As the ionic liquid, a hydrophilic ionic liquid is preferably used. In the present specification, the term “hydrophilic” means capable of being dissolved or completely dispersed in an aqueous solvent.

Examples of the cation of the hydrophilic ionic liquid include a quaternary ammonium cation, a phosphonium cation, a pyridinium cation, and a piperidinium cation.

Examples of the anion of the hydrophilic ionic liquid include a phosphate ion, an acetate ion, a citrate ion, a tetrafluoroborate ion, and hexafluorophosphoric acid.

More specifically, examples of the hydrophilic ionic liquid include choline dihydrogen phosphate, choline dihydrogen citrate, 1-ethylpyridinium tetrafluoroborate, 1-butylpyridinium bromide, 1-butyl-4-methylpyridinium tetrafluoroborate, 1-(3-cyanopropyl) pyridinium chloride, 1-butyl-1-methylpiperidinium hexafluorophosphate, and choline acetate.

The ionic liquid contained in the sealing liquid 22 and the internal liquid 22a may contain one or more types of cations and one or more types of anions.

When the ionic liquid contained in the sealing liquid 22 and the internal liquid 22a contains the above-described cations, the sealing liquid 22 and the internal liquid 22a may contain another anion other than the above-described anions.

Examples of the other anion include the above-described inorganic anion and a polyatomic anion.

When the ionic liquid contains the above-described cations, preferably, the sealing liquid 22 or the internal liquid 22a does not contain the above-described inorganic cation. This makes it possible to prevent the inorganic cation contained in the internal liquid 22a from passing through the gap 18 to flow out to the external liquid 22b.

As a result, the inorganic cation contained in the external liquid 22b can be suppressed from passing through the gap 18 to flow into the internal liquid 22a. This makes it possible to more accurately measure the amount of inorganic cations flowing from the external liquid 22b into the internal liquid 22a through pores of the membrane protein 31 of the lipid bimolecular membrane 30.

In addition, when the ionic liquid contained in the sealing liquid 22 and the internal liquid 22a contains the above-described anions, the sealing liquid 22 and the internal liquid 22a may contain another cation other than the above-described cations.

Examples of the other cation include the above-described inorganic cation and a polyatomic cation.

When the ionic liquid contained in the sealing liquid 22 and the internal liquid 22a contains the above-described anions, preferably, the sealing liquid 22 or the internal liquid 22a does not contain the above-described inorganic anion. This makes it possible to prevent the inorganic anion contained in the internal liquid 22a from passing through the gap 18 to flow out to the external liquid 22b.

As a result, the inorganic anion contained in the external liquid 22b can be suppressed from passing through the gap 18 to flow into the internal liquid 22a. This makes it possible to more accurately measure the amount of inorganic anions passing through pores of the membrane protein 31 of the lipid bimolecular membrane 30 to flow from the external liquid 22b into the internal liquid 22a.

As illustrated in FIG. 2, the lipid bimolecular membrane substrate 1A includes the electrode unit 40 and the counter electrode unit 41. The electrode unit 40 includes an electrode 40a in contact with the internal liquid 22a filling the inside of the microwell 20, and a conductive wire 40b connected to the electrode 40a. The electrode 40a is disposed on an inner peripheral surface of the microwell 20.

The counter electrode unit 41 of the lipid bimolecular membrane substrate 1A includes a counter electrode 41a in contact with the external liquid 22b covering the outside of the microwell 20, and a conductive wire 41b connected to the counter electrode 41a.

Examples of materials of the electrode 40a and the counter electrode 41a include silver/silver chloride, gold, and platinum.

The electrode unit 40 and the electrode unit 41 are connected to a measuring device, and a current (ion flow) or the like passing through pores of the membrane protein 31 of the lipid bimolecular membrane 30 is measured, whereby a function of the membrane protein 31 introduced into the lipid bimolecular membrane 30 can be analyzed electrochemically.

The lipid bimolecular membrane substrate 1A does not have to include the electrode unit 40. In this case, the amount of ions passing through pores of the membrane protein 31 can be measured using the fluorescent molecule 23 contained in the internal liquid 22a of the microwell 20.

The lipid bimolecular membrane substrate 1A does not have to include the electrode unit 41. In this case, a separately-prepared electrode is brought into contact with the external liquid 22b of the microwell 20, and this electrode and the electrode unit 40 are connected to a measuring device, whereby a potential difference and a current can be measured.

By using the sealing liquid 22 containing the ionic liquid, it is possible to more accurately measure, for example, the amount of ions passing through pores of the membrane protein 31 of the lipid bimolecular membrane 30 using the electrode unit 40 and the counter electrode unit 41.

### Second Embodiment

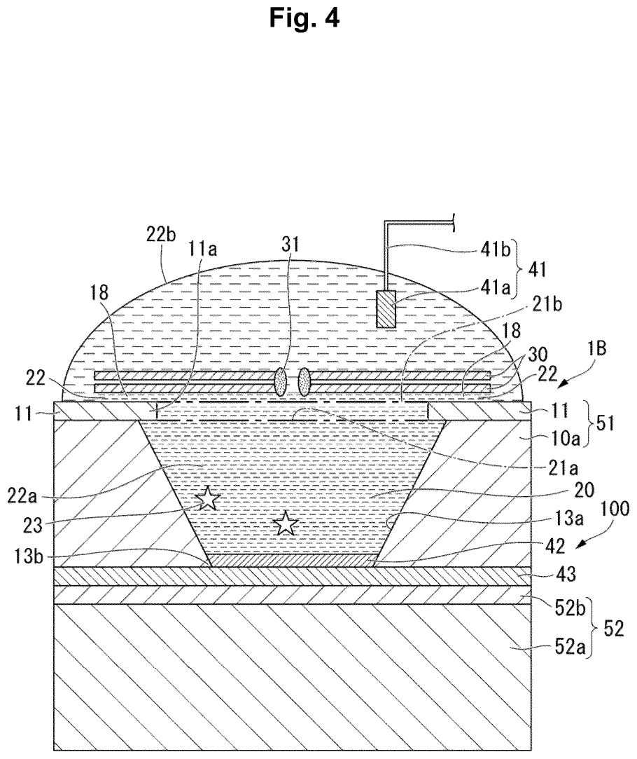

FIG. 4 illustrates a cross-sectional view of a lipid bimolecular membrane substrate 1B according to an embodiment.

As illustrated in FIG. 4, the lipid bimolecular membrane substrate 1B includes a substrate 100 having a first layer 51 and a second layer 52. The substrate 100 includes an electrode 43 sandwiched between the first layer 51 and the second layer 52.

In the lipid bimolecular membrane substrate 1B, the same portions as those of the lipid bimolecular membrane substrate 1A of the first embodiment are denoted by the same reference numerals, and description thereof is omitted.

The first layer 51 of the lipid bimolecular membrane substrate 1B includes a substrate body 10a in which a through-hole 13a passing through the substrate body 10a in a thickness direction is formed. The first layer 51 does not have to have a thin film layer 11.

The second layer 52 includes a substrate body 52a of the second layer and an insulating layer 52b laminated on the substrate body 52a of the second layer 52.

A material of the insulating layer 52b is not particularly limited, and examples thereof include silicon, a silicon oxide, a silicon nitride, quartz, mica, glass, and plastic. When the substrate body 52a is insulating, examples of a material of the substrate body 52a include the above-described insulating materials.

A method for forming the insulating layer 52b may be, for example, a method for sputtering the material of the insulating layer 52b on the substrate body 52a of the second layer.

When the insulating layer 52b is made of a silicon oxide, the insulating layer 52b may be formed on the substrate body 52a of the second layer 52 by a thermal oxidation method using the substrate body 52a made of silicon.

The first layer 51 has the through-hole 13a passing through the first layer 51 in the thickness direction. The electrode 43 is exposed at a lower end of the through-hole 13a.

A space surrounded by the through-hole 13a, an overhang portion 11a, an opening 21b of the thin film layer 11, and the electrode 43 forms a microwell 20.

The electrode 43 is plated with a plating layer 42. A part of the plating layer 42 rides on a lower end portion 13b of the through-hole 13a. The electrode 43 does not have to have the plating layer 42.

A metal element contained in the plating layer 42 is not particularly limited as long as the metal element has a high electrical conductivity and can form a plating liquid, and examples thereof include silver/silver chloride, gold, and platinum.

The electrode 43 having the plating layer 42 on a surface thereof is preferably a non-polarized electrode.

Examples of the non-polarized electrode include an electrode made of silver and chlorinated.

The plating layer 42 has a larger surface roughness than a surface of the electrode 43 that is not plated, and has an increased surface area. Therefore, the electrode 43 on which the plating layer 42 is formed is superior in measurement sensitivity to the electrode 43 that is not plated.

The lipid bimolecular membrane substrates 1A and 1B described above can suppress ion leakage through a thin interfacial aqueous layer present between the substrate 12 and the lipid bimolecular membrane 30.

An ion flow passing through a channel of the membrane protein 31 is weak. Therefore, in the conventional technique, it is difficult to determine whether an obtained response is a channel signal derived from a membrane protein or due to ion leakage.

Meanwhile, according to the present invention, since the ion leakage is suppressed, it is possible to measure a function of the membrane protein 31 at a molecular level. In addition, since an analysis method using the lipid bimolecular membrane substrates 1A and 1B is simpler than a patch clamp method, a function of a target protein can be specifically measured.

In addition, by arraying the microwells 20 using a semiconductor technique, for example, application to high-throughput screening in a field of drug discovery is also expected.

[Manufacturing Method]

### First Embodiment

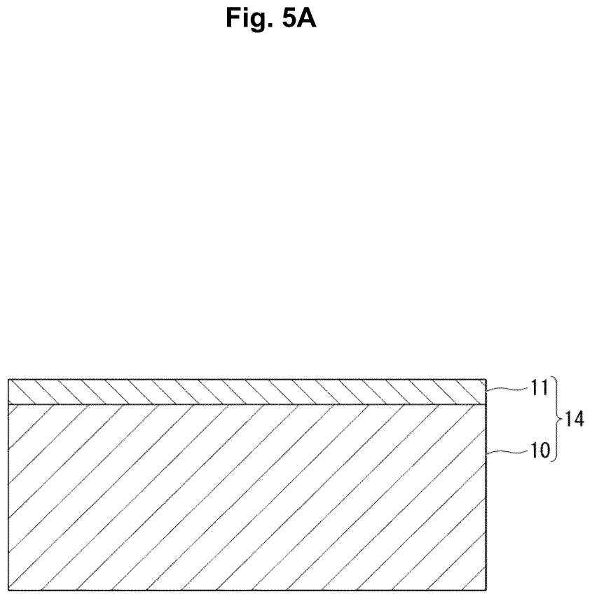

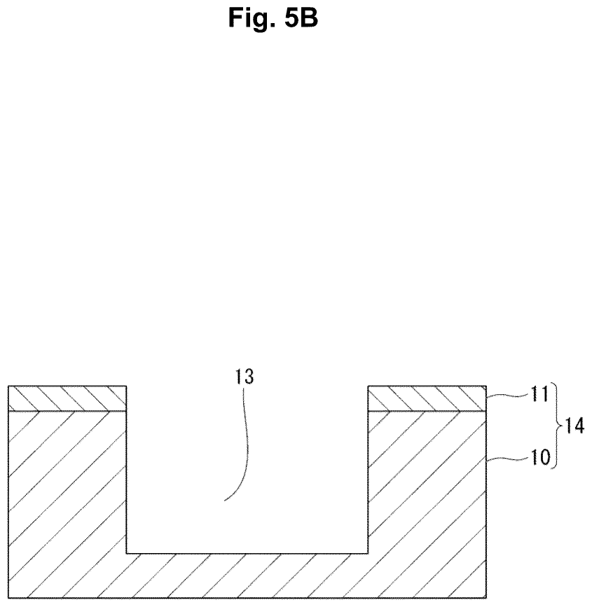

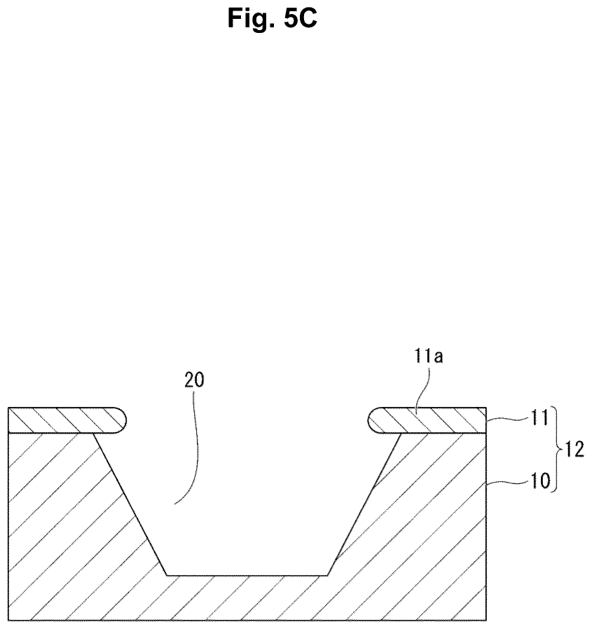

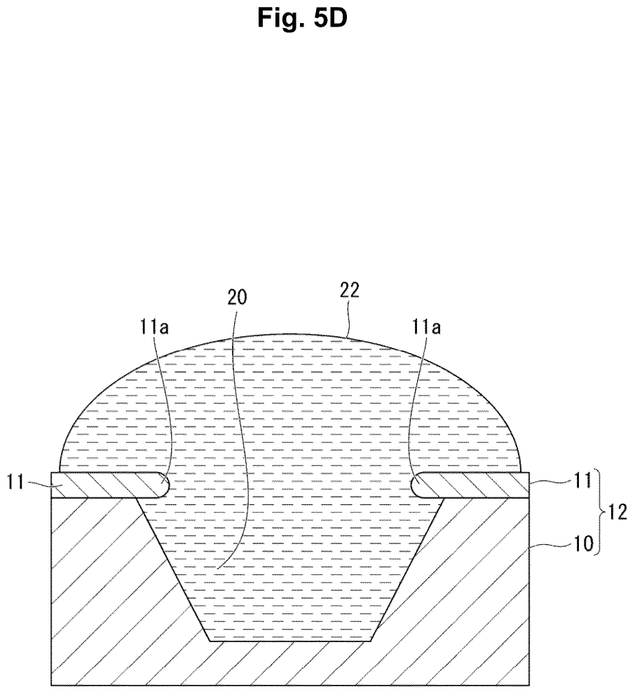

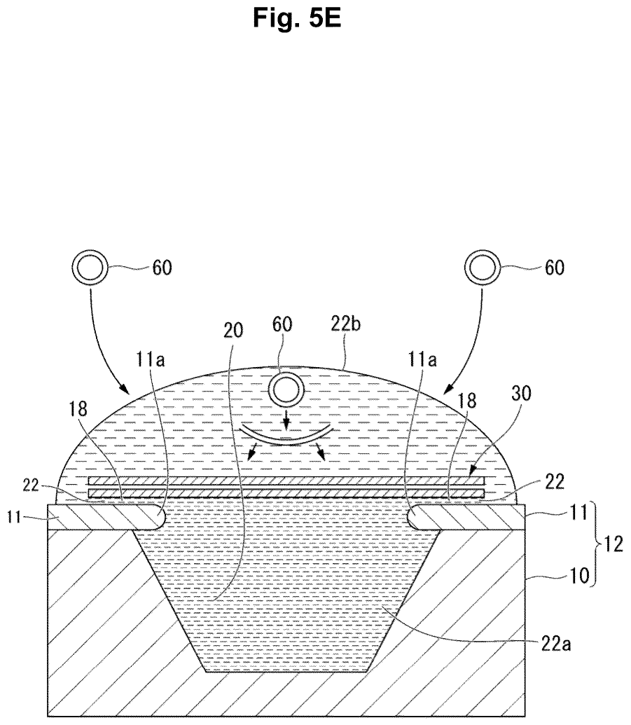

FIGS. 5A to 5E are process diagrams illustrating a method for manufacturing the lipid bimolecular membrane forming substrate 1A illustrated in FIG. 2. The method for manufacturing the lipid bimolecular membrane substrate 1A includes the following steps (a) to (f).

Step (a): a step of forming the thin film layer 11 on the substrate body 10 to obtain a laminate 14

Step (b): a step of etching the laminate 14 to form the recess 13 in the substrate body 10

Step (c): a step of etching the substrate body 10 in the recess 13

Step (d): a step of dropping a sealing liquid 22 onto a substrate 12, filling the inside of the microwell 20 with the sealing liquid 22, and covering a peripheral edge of the microwell 20 with the sealing liquid 22

Step (e): a step of adding the lipid membrane vesicle 60 to the sealing liquid 22 on the microwell 20

Step (f): a step of developing the lipid membrane vesicle 60 in the sealing liquid 22 to cover an opening of the microwell 20 with the lipid bimolecular membrane 30

Hereinafter, each of the steps will be described in detail.

<Step (a)>

As illustrated in FIG. 5A, in step (a), the thin film layer 11 is formed on the substrate body 10 to obtain the laminate 14.

Examples of materials of the substrate body 10 and the thin film layer 11 include those described above.

A method for forming the thin film layer 11 may be, for example, a method for sputtering the material of the thin film layer 11 on the substrate body 10. Alternatively, the method for forming the thin film layer 11 may be a method for forming the thin film layer 11 of a silicon oxide film on a silicon substrate by a thermal oxidation method when the substrate body 10 is a silicon substrate.

<Step (b)>

As illustrated in FIG. 5B, in step (b), the laminate 14 is etched to form the recess 13 in the substrate body 10.

A method for etching the laminate 14 is not particularly limited, and examples thereof include a method in which an electron beam lithography method, a photolithography method, and a dry etching method are combined.

<Step (c)>

As illustrated in FIG. 5C, in step (c), the substrate body 10 is etched in the recess 13. Examples of a method for etching the substrate body 10 include a wet etching method.

Examples of a solution used in the wet etching method include an alkali solution such as a sodium hydroxide solution or a potassium hydroxide solution.

The substrate body 10 has a higher etching rate than the thin film layer 11. Therefore, the overhang portion 11a protruding in a direction of narrowing the opening 21a of the recess 13 is formed. In this way, the microwell 20, which is a space surrounded by the recess 13, the thin film layer 11, and the opening 21b of the thin film layer 11, is obtained.

<Step (d)>

As illustrated in FIG. 5D, in step (d), the sealing liquid 22 is dropped onto the substrate 12, the inside of the microwell 20 is filled with the sealing liquid 22, and a peripheral edge of the microwell 20 is covered with the sealing liquid 22.

The sealing liquid 22 contains an ionic liquid. Examples of the ionic liquid include those described above.

<Step (e)>

As illustrated in FIG. 5E, in step (e), the above-described lipid membrane vesicle 60 is added to the external liquid 22b on the microwell 20.

Examples of a representative method for forming the lipid membrane vesicle 60 include a static hydration method (for example, Akashi K et al., Preparation of giant liposomes in physiological conditions and their characterization under an optical microscope, Biophys J. 1996.; 71: 3242-3250) and an electric field forming method (for example, Dimitrov D S and Angelova M I, Lipid swelling and liposome formation mediated by electric fields, 1998; 19: 323-336).

As the method for forming the lipid membrane vesicle 60, an electric field forming method is preferably adopted. In the electrolysis forming method, it is easy to prepare the lipid membrane vesicle 60, and a reaction time and a reaction process are simple. The electric field forming method is a method in which a thin phospholipid film is formed on an electrode such as indium tin oxide (ITO), and then an alternating electric field is applied to form a giant lipid membrane vesicle in an aqueous solution.

The lipid membrane vesicle 60 has a size that can sufficiently cover the opening 21b of one or more microwells 20 formed in advance on the substrate when being developed on the microwell 20 of the substrate 12 to form the lipid bimolecular membrane 30 on a plane.

The lipid membrane vesicle 60 has a diameter (major axis) of preferably 1 μm or more, more preferably 3 μm or more, still more preferably 5 μm or more, particularly preferably 10 μm or more. As a result, the lipid bimolecular membrane 30 formed by development can reliably cover the opening 21b of the microwell 20.

The lipid membrane vesicle 60 has a diameter of preferably 100 μm or less, more preferably 50 μm or less, still more preferably 30 μm or less. This makes it possible to maintain the structure of the lipid membrane vesicle 60.

The diameter of the lipid membrane vesicle is measured by a general optical observation method.

In order to obtain lipid membrane vesicles having a uniform size, it is preferable to form a uniform phospholipid molecule membrane having a thickness of several tens nm to several μm on an ITO substrate, and it is preferable to apply an alternating electric field of about several hundred mV to 2 V.

<Step (f)>

As illustrated in FIG. 5E, in step (f), the lipid membrane vesicle 60 is developed in the sealing liquid 22 to cover the opening 21b of the microwell 20 with the lipid bimolecular membrane 30.

After a lapse of a certain time after step (e), the lipid membrane vesicle 60 is cleaved in the sealing liquid 22 to form a lipid bimolecular membrane.

When a material of the thin film layer 11 is a silicon oxide, a silicon nitride, or the like, and the sealing liquid 22 is a neutral solution, the thin film layer 11 has a negative surface charge.

The lipid bimolecular membrane formed by cleavage is attracted to a surface of the thin film layer 11 by electrostatic attraction, and the opening 21b of the microwell 20 is covered with the lipid bimolecular membrane 30.

By step (f), the gap 18 between the substrate 12 and the lipid bimolecular membrane 30 is sealed with the sealing liquid 22.

The certain time is not particularly limited, but is preferably, for example, 30 seconds to 10 minutes. As a result, the opening 21b of the microwell 20 can be reliably covered with the lipid bimolecular membrane 30.

When the opening 21b of the microwell 20 is covered with the lipid bimolecular membrane 30, the lipid bimolecular membrane 30 covering the microwell 20 is covered with the sealing liquid 22. The composition of the internal liquid 22a inside the microwell 20 is the same as that of the sealing liquid 22.

The sealing liquid 22 covering the opening 21b of the microwell 20 may be removed, a solution different from the sealing liquid 22 may be dropped onto the substrate 12, and the microwell 20 may be covered with the external liquid 22b. Examples of the external liquid 22b include those described above.

### Second Embodiment

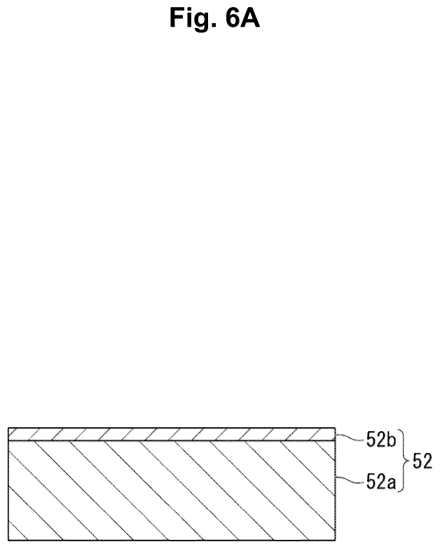

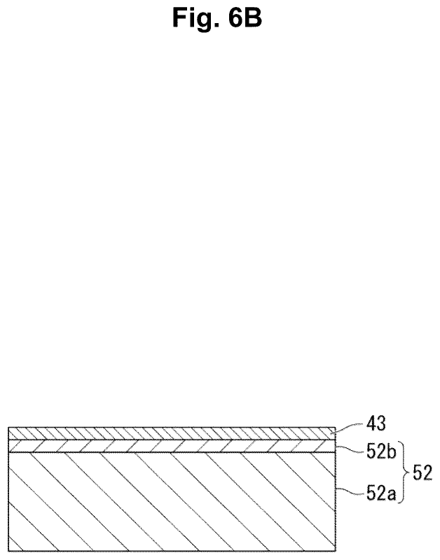

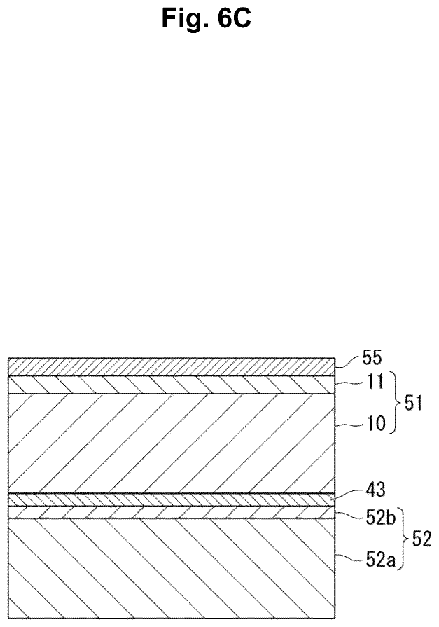

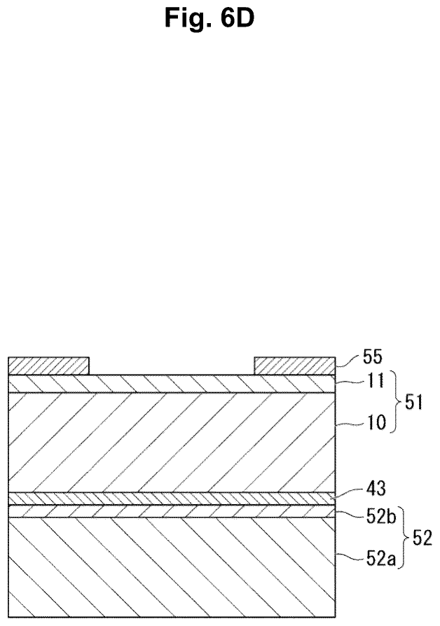

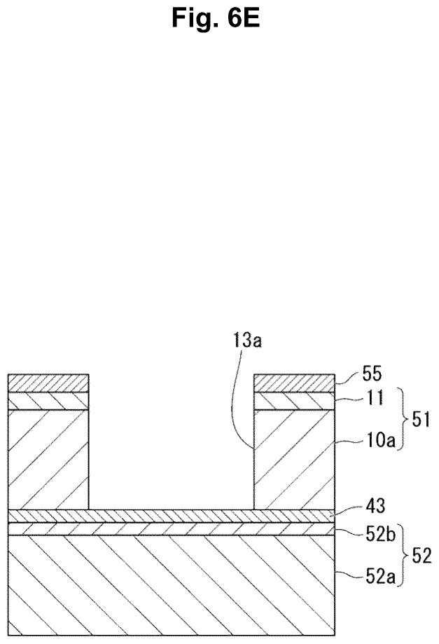

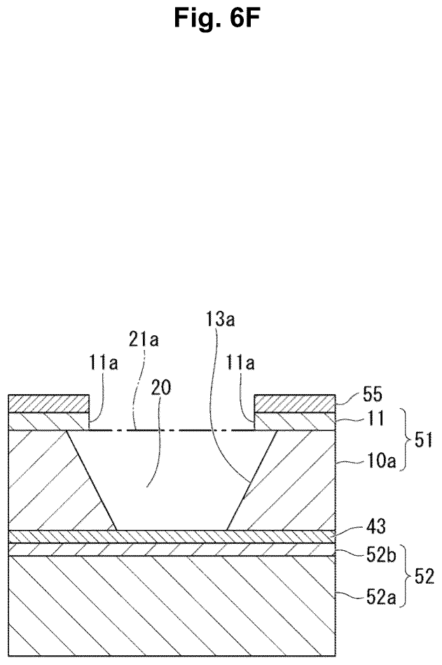

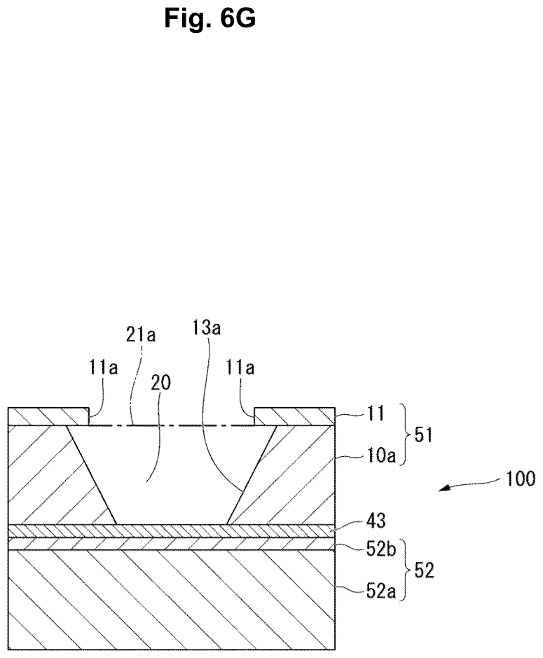

FIGS. 6A to 6G are process diagrams illustrating a method for manufacturing the lipid bimolecular membrane forming substrate 1B illustrated in FIG. 4. The method for manufacturing the lipid bimolecular membrane substrate 1B includes the following steps (i) to (x) prior to the above steps (d) to (f).

Step (i): a step of laminating the insulating layer 52b on the substrate body 52a to obtain the second layer 52

Step (ii): a step of laminating the electrode 43 on the second layer 52

Step (iii): a step of forming the substrate body 10 on the electrode 43

Step (iv): a step of laminating the thin film layer 11 on the substrate body 10 to form the first layer 51 including the substrate body 10 and the thin film layer 11 on the electrode 43

Step (v): a step of forming the resist film 55 on the thin film layer 11

Step (vi): a step of etching the resist film 55 to form a resist pattern

Step (vii): a step of forming the through-hole 13a in the first layer 51

Step (viii): a step of etching the substrate body 10 to form the microwell 20 surrounded by the through-hole 13a, the overhang portion 11a, the opening 21a of the through-hole 13a, and the electrode 43 exposed in the through-hole 13a

Step (ix): a step of removing the resist film 55

Step (x): a step of forming the plating layer 42 on a surface of the electrode 43 which is a bottom surface of the microwell 20

Hereinafter, each of the steps will be described in detail.

<Step (i)>

As illustrated in FIG. 6A, in step (i), the insulating layer 52b is laminated on the substrate body 52a to obtain the second layer 52.

Examples of a material of the substrate body 50 include the same materials as the above-described materials of the substrate body 10. Examples of a material of the insulating layer 52b include the same material as the material of the thin film layer 11. Examples of a method for forming the insulating layer 52b include the same method as the method for forming the thin film layer 11.

<Step (ii)>

As illustrated in FIG. 6B, in step (ii), the electrode 43 is laminated on the second layer 52.

Examples of a material of the electrode 43 include gold, platinum, silver, and copper.

A method for forming the electrode 43 may be a sputtering method or a method for plating a surface of the second layer 52 with a metal material.

<Step (iii)>

As illustrated in FIG. 6C, in step (iii), the substrate body 10 is laminated on the electrode 43.

Examples of a material of the substrate body 10 include those described above. Examples of a method for forming the substrate body 10 on the electrode 43 include a sputtering method and a plasma method.

<Step (iv)>

As illustrated in FIG. 6C, in step (iv), the thin film layer 11 is laminated on the substrate body 10 to form the first layer 51 including the substrate body 10 and the thin film layer 11 on the electrode 43.

Examples of a material of the thin film layer 11 include those described above. Examples of a method for forming the thin film layer 11 include the methods described above.

<Step (v)>

As illustrated in FIG. 6C, in step (v), the resist film 55 is formed on the thin film layer 11.

As a material of the resist film 55, a material known to those skilled in the art can be used. A method for forming the resist film 55 may be a method known to those skilled in the art.

<Step (vi)>

As illustrated in FIG. 6D, in step (vi), the resist film 55 is etched to form a resist pattern. An etching method may be a photolithography method or an electron beam lithography method.

<Step (vii)>

As illustrated in FIG. 6E, in step (vii), the through-hole 13a is formed in the first layer 51 having the resist film 55 on which the resist pattern is formed by a dry etching method or an electron beam lithography method.

<Step (viii)>

As illustrated in FIG. 6F, in step (viii), the microwell 20 surrounded by the through-hole 13a, the overhang portion 11a, the opening 21a of the through-hole 13a, and the electrode 43 exposed in the through-hole 13a is formed by a wet etching method.

The substrate body 10a has a higher etching rate than the thin film layer 11. Therefore, the overhang portion 11a protruding in a direction of narrowing the opening 21a of the through-hole 13a is formed.

<Step (ix)>

As illustrated in FIG. 6G, in step (ix), the resist film 55 is removed. As a method for removing the resist film 55, a method known to those skilled in the art can be used.

<Step (x)>

In step (x), the plating layer 42 is formed on a surface of the electrode 43 in the through-hole 13a.

Examples of the plating layer 42 include those described above. Examples of a method for forming the plating layer 42 include an electroplating method.

In the electroplating method, specifically, first, a plating liquid is dropped onto the microwell 20. As the plating liquid, an electrolytic solution known to those skilled in the art may be used.

Subsequently, a current is applied in a state where a plating metal is immersed in the plating liquid to deposit the plating metal on a surface of the electrode 43. Examples of the plating metal include copper, silver, gold, and platinum.

When silver is used as the plating metal, after silver plating is performed, silver plating may be chlorinated. As a result, the electrode 43 in the through-hole 13a can be formed into a non-polarized electrode.

### Examples

Hereinafter, the present invention will be described with reference to Examples, but the present invention is not limited to the following Examples. In the present specification, the unit “M” means “mol/L”.

### Experimental Example 1

(Manufacture of Lipid Bimolecular Membrane Substrate)

A lipid membrane vesicle was developed on a substrate having a microwell using an ionic liquid as a sealing liquid to obtain a lipid bimolecular membrane substrate in which an opening of the microwell was covered with a lipid bimolecular membrane.

<Substrate>

As a substrate body, a silicon substrate was used. Subsequently, as a thin film layer, a silicon oxide film layer having a thickness of 120 nm was formed on an upper surface of the silicon substrate by a thermal oxidation method.

Subsequently, a recess having a circular opening (diameters of 2 μm and 4 μm) was formed using a photolithography method and a dry etching method.

Subsequently, the substrate body under the thin film layer was selectively etched using a potassium hydroxide aqueous solution (10% by mass) to form an overhang portion of the thin film layer. As a result, a substrate having a microwell was obtained.

<Preparation of Giant Lipid Membrane Vesicle>

First, a solution was prepared using diphytanyl phosphatidylcholine (DPhPC) (80 mol %), cholesterol (20 mol %), and chloroform which become a neutral charged state around pH 7.

To the obtained solution, rhodamine-dipalmitoylphosphatidylethanolamine (Rhod-DOPE) was added in an amount of 0.05 mol % with respect to the total amount (100 mol %) of lipids contained in the solution to prepare a mixed lipid solution.

Subsequently, 200 μL of the mixed chloroform solution was uniformly applied onto an ITO substrate (substrate with an ITO thin film formed on SiO₂, size 40×40 mm).

This substrate was dried under reduced pressure at room temperature for two hours to completely remove the chloroform solvent, thereby forming a uniform lipid molecular thin film on the ITO substrate. A silicone rubber having a thickness of 1 mm and having a window portion obtained by hollowing a silicone rubber having an outer dimension of 30×30 mm and a thickness of 1 mm at a size of 20×20 mm was disposed on the lipid molecular thin film in close contact, and 500 μL of a 200 mM sucrose aqueous solution was dropped onto the window portion.

Furthermore, an ITO substrate was disposed on the silicone rubber such that air bubbles were not included, and the solution in the silicone rubber window portion was sandwiched between the ITO substrates. Subsequently, a clip electrode was bonded to the ITO substrates, and an alternating electric field was applied in a thermostatic bath to obtain a sucrose dispersion of a lipid membrane vesicle by an electric field forming method.

<Development of Lipid Membrane Vesicle to Substrate>

Onto the substrate having a microwell, 100 μL of an ionic liquid aqueous solution (mixed solution of 160 mM glucose, 20 μM calcein, 1 mM EDTA, and 20 mM choline dihydrogen phosphate) was dropped as a sealing liquid.

The sucrose dispersion of the lipid membrane vesicle was dropped onto the substrate and allowed to stand for five minutes to develop the lipid membrane vesicle on the substrate, thereby covering an opening of a microwell of the substrate with a lipid bimolecular membrane. As a result, a gap between the substrate and the lipid bimolecular membrane was sealed with the sealing liquid.

At this time, an internal liquid of the microwell was filled with the sealing liquid, and the microwell and the lipid bimolecular membrane were covered with the sealing liquid (external liquid).

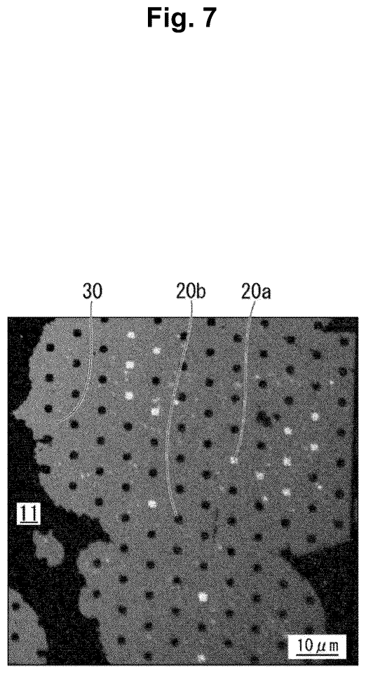

Subsequently, the sealing liquid was removed from the external liquid covering an upper portion of the microwell, and the external liquid of the well portion was replaced with a 200 mM glucose solution. Results thereof are illustrated in FIG. 7.

FIG. 7 is a photograph of fluorescence of calcein taken with a fluorescence microscope. In FIG. 7, reference numeral 30 denotes a lipid bimolecular membrane developed on a substrate, reference numeral 20a denotes a microwell in which calcein fluorescence was observed, and reference numeral 20b denotes a microwell in which calcein fluorescence was not observed. In FIG. 7, reference numeral 11 denotes a thin film layer, and reference numeral 30 denotes a lipid bimolecular membrane developed on a thin film layer.

Reference numeral 20a indicates that calcein is confined inside a microwell because an opening of the microwell is covered with a lipid bimolecular membrane. Reference numeral 20b indicates that calcein is not confined inside a microwell because an opening of the microwell is not covered with a lipid bimolecular membrane.

### Experimental Example 2

(Suppression of Ion Leakage by Ionic Liquid)

Two types of lipid bimolecular membrane substrates were prepared in a similar manner to Experimental Example 1 except that an inorganic ionic aqueous solution (mixed solution of 200 mM glucose, 20 μM fluo-4, 1 mM EDTA, and 20 mM potassium chloride) or an ionic liquid aqueous solution (mixed solution of 160 mM glucose, 20 μM fluo-4, 1 mM EDTA, and 20 mM choline dihydrogen phosphate) was used as a sealing liquid. Here, fluo-4 is a calcium fluorescent probe.

Subsequently, a calcium chloride aqueous solution was dropped onto an external liquid covering a microwell of each of the lipid bimolecular membrane substrates. By observing fluorescence of fluo-4 in the microwell, a state in which a calcium ion contained in the external liquid passed through a gap to flow into an internal liquid in each of the obtained two types of lipid bimolecular membrane substrates was monitored.

Here, the inventors have presumed as follows.

It is known that an interfacial aqueous layer having a thickness of 1 to 2 nm is formed between a lipid bimolecular membrane and a substrate (hereinafter, also simply referred to as “gap”). A potassium ion has an ionic radius of about 0.15 nm, and it is considered that the potassium ion can easily pass through the gap.

The chemical structure of choline dihydrogen phosphate is indicated below. A cation and an anion of choline dihydrogen phosphate are bulky as compared with an inorganic ion. It is considered that a choline ion, which is a cation, has difficulty in passing through the gap.

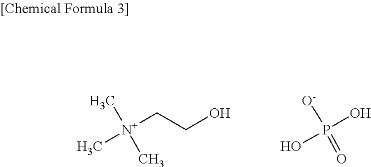

In order for a calcium ion contained in the external liquid of the microwell to pass through the gap to flow into the internal liquid of the microwell, a cation contained in the internal liquid of the microwell needs to pass through the gap to flow out from the internal liquid of the microwell to the external liquid thereof, and the internal liquid of the microwell and the external liquid thereof need to maintain electrical neutrality.

When a potassium chloride aqueous solution is used as a sealing liquid, a potassium ion can easily pass through the gap to move from the internal liquid of the microwell to the external liquid thereof. Therefore, a calcium ion contained in the external liquid of the microwell can pass through the gap to flow into the internal liquid of the microwell.

Meanwhile, when a choline dihydrogen phosphate aqueous solution is used as a sealing liquid, it is difficult for choline to pass through the gap, and it is difficult for choline to move from the internal liquid of the microwell to the external liquid thereof. Therefore, a calcium ion contained in the external liquid of the microwell is suppressed from passing through the gap to flow into the internal liquid of the microwell.

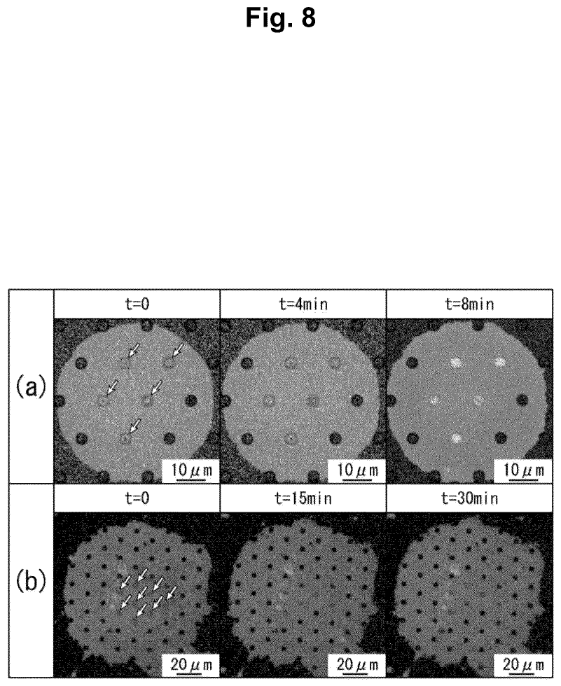

FIG. 8 illustrates a result of measuring a temporal change in the fluorescence intensity of fluo-4 inside the microwell.

In FIG. 8, (a) is a result when an inorganic ion aqueous solution is used as a sealing liquid, and (b) is a result when an ionic liquid aqueous solution is used as a sealing liquid. In FIG. 8, t represents a time elapsed since a calcium chloride aqueous solution was dropped, and an arrow represents a microwell covered with a lipid bimolecular membrane.

Immediately after the calcium chloride aqueous solution was added (t=0), the fluorescence intensity hardly changed in each of the lipid bimolecular membrane substrates. That is, it was confirmed that calcium did not flow from the external liquid of the microwell into the internal liquid thereof immediately after the calcium chloride aqueous solution was added to the external liquid of the microwell.

In the lipid bimolecular membrane substrate using an inorganic ion aqueous solution as a sealing liquid, fluorescence of fluo-4 was observed from a microwell covered with a lipid bimolecular membrane after about eight minutes.

This result indicates that in the lipid bimolecular membrane substrate using an inorganic ion aqueous solution as a sealing liquid, a calcium ion contained in the external liquid covering the lipid bimolecular membrane passed through the gap between the lipid bimolecular membrane and the substrate to flow into the microwell.

Meanwhile, in the lipid bimolecular membrane substrate using an ionic liquid aqueous solution as a sealing liquid, a large change in fluorescence intensity was not observed even after 30 minutes or more.

This result indicates that in the lipid bimolecular membrane substrate using an ionic liquid aqueous solution as a sealing liquid, a calcium ion contained in the external liquid covering the lipid bimolecular membrane does not flow into the microwell through the gap between the lipid bimolecular membrane and the substrate, or flow into the microwell is significantly suppressed.

That is, it has been revealed that movement of ions through the gap between the lipid bimolecular membrane and the substrate is reduced by sealing the gap between the lipid bimolecular membrane and the substrate with a sealing liquid containing an ionic liquid.

It has been revealed that the lipid bimolecular membrane substrate of the present invention using a sealing liquid containing an ionic liquid is suitable in order to introduce a membrane protein into a lipid bimolecular membrane substrate, to reduce noise of measurement current and measurement potential, and to measure a function of the membrane protein with high sensitivity.

### Experimental Example 3

(Manufacture of Lipid Bimolecular Membrane Substrate Including Electrode)

First, a silicon wafer was used as a substrate body of a second layer, and covered with a thermal oxide film (insulating layer of the second layer) having a thickness of 120 nm by a thermal oxidation method to obtain the second layer.

Subsequently, a gold layer was deposited on the second layer so as to have a thickness of 60 nm and patterned by a photolithography method to form an electrode having a thickness of about 60 nm.

This electrode is connected to a sufficiently large pad portion (300 μm square), and can be connected to an electrophysiology measuring device such as a patch clamp measuring device by normal wire bonding.

A silicon nitride film was deposited on the electrode so as to have a thickness of 1 μm by a plasma CVD method to laminate a substrate body of a first layer. Subsequently, a silicon oxide film (thin film layer of the first layer) having a thickness of 200 nm was deposited on the substrate body of the first layer by a sputtering method to obtain a substrate having the first layer and the second layer.

Subsequently, a resist film was laminated on the first layer, and then a resist pattern was formed by a photolithography method. Subsequently, a through-hole was formed in the first layer by a dry etching method to expose a surface of the electrode.

Subsequently, an overhang portion was formed in the thin film layer of the first layer by a wet etching method using selectivity between the substrate body of the first layer and the thin film layer of the first layer. Subsequently, the resist film was washed and removed.

As a result, a substrate in which a microwell surrounded by the through-hole of the first layer and the electrode exposed in the through-hole was formed was obtained.

A lipid membrane vesicle was developed on the obtained substrate using a sealing liquid containing an ionic liquid to cover an opening of the microwell with a lipid bimolecular membrane, thereby obtaining a lipid bimolecular membrane substrate.

A membrane protein can be introduced into the lipid bimolecular membrane covering the microwell of the obtained lipid bimolecular membrane substrate by a vesicle fusion method or the like. In addition, by disposing a counter electrode on the lipid bimolecular membrane substrate and measuring a current flowing between the electrode and the counter electrode when a voltage is applied between the electrode and the counter electrode, an ion current through the membrane protein can be detected.

### Experimental Example 4

(Manufacture of Lipid Bimolecular Membrane Substrate Including Electrode Having Plating Layer)

Using the substrate including an electrode, obtained in Experimental Example 3, the electrode was plated to prepare a substrate including a non-polarized electrode.

Onto the substrate including an electrode, obtained in Experimental Example 3, 200 μL of a silver plating liquid (PRECIOUSFAB Ag4710, TANAKA PRECISION METAL) was dropped.

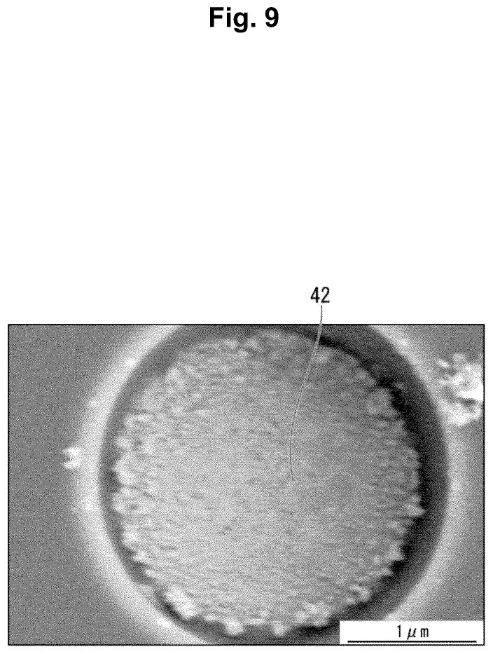

A silver wire connected to a manual prober was put in the plating liquid, and the electrode was connected to the manual prober. A constant current (0.1 to 0.5 nA) was applied between the silver wire and the electrode for one minute to plate a surface of the electrode with silver. Thereafter, the silver surface was immersed in a chlorine-based bleaching liquid for two minutes to chlorinate the silver surface, thereby forming a silver/silver chloride electrode. Results thereof are illustrated in FIG. 9.

FIG. 9 is a photograph of a bottom surface of a microwell taken with an electron microscope. It has been revealed that a silver/silver chloride electrode unit is formed on the bottom surface of the microwell. In addition, since the silver/silver chloride surface has irregularities and a surface area thereof is increased, it has been revealed that the silver/silver chloride surface has a more preferable shape in electrophysiological measurement.

With this substrate including a non-polarized electrode, a lipid bimolecular membrane substrate was prepared using a sealing liquid containing an ionic liquid in a similar manner to Experimental Example 1.

In the obtained lipid bimolecular membrane substrate, a membrane protein can be introduced into the lipid bimolecular membrane covering the microwell by a vesicle fusion method or the like. In addition, by disposing a counter electrode on the lipid bimolecular membrane substrate and measuring a current flowing between the non-polarized electrode and the counter electrode when a voltage is applied between the non-polarized electrode and the counter electrode, an ion current through the membrane protein can be detected with high sensitivity.

### INDUSTRIAL APPLICABILITY

Provided are a lipid bimolecular membrane substrate suppressing ion leakage and a method for manufacturing the lipid bimolecular membrane substrate. This lipid bimolecular membrane substrate is suitably used for analyzing a function of a membrane protein.

### REFERENCE SIGNS LIST

1A, 1B Lipid bimolecular membrane substrate
10, 10a Substrate body
11 Thin film layer
11a Overhang portion
12 Substrate
13 Recess
13a Through-hole
13b End portion
14 Laminate
18 Gap
20 Microwell
21a, 21b Opening
22 Sealing liquid
22a Internal liquid
22b External liquid
23 Fluorescent molecule
30 Lipid bimolecular membrane
31 Membrane protein
40 Electrode unit
40a Electrode
40b Conductive wire
41 Counter electrode unit
41a Counter electrode
41b Conductive wire
43 Electrode layer
51 First layer
52 Second layer
52a Substrate body
52b Insulating layer
55 Resist film
60 Lipid membrane vesicle
100 Substrate

1. A lipid bimolecular membrane substrate comprising:
a substrate in which a microwell opened on one surface is formed;
a lipid bimolecular membrane disposed on the substrate so as to cover an opening of the microwell; and
a sealing liquid disposed between the substrate and the lipid bimolecular membrane, wherein
the sealing liquid contains an ionic liquid.

2. The lipid bimolecular membrane substrate according to claim 1, wherein an electrode is disposed on an inner peripheral surface of the microwell.

3. The lipid bimolecular membrane substrate according to claim 2, wherein the electrode has a plating layer on a surface thereof.

4. The lipid bimolecular membrane substrate according to claim 2, wherein
the substrate includes a first layer, a second layer, and the electrode sandwiched between the first layer and the second layer,
the first layer has a through-hole passing through the first layer in a thickness direction,
the electrode is exposed at a lower end of the through-hole, and
a space surrounded by the through-hole and the electrode constitutes the microwell.

5. The lipid bimolecular membrane substrate according to claim 4, wherein the second layer has an insulating layer.

6. A method for manufacturing a lipid bimolecular membrane substrate, the method comprising:
dropping a sealing liquid onto a substrate in which a microwell opened on one surface is formed, filling an inside of the microwell with the sealing liquid, and covering a peripheral edge of the microwell with the sealing liquid;
adding a lipid membrane vesicle to the sealing liquid on the microwell; and
developing the lipid membrane vesicle in the sealing liquid to cover an opening of the microwell with a lipid bimolecular membrane, wherein
the sealing liquid contains an ionic liquid.

7. The method for manufacturing a lipid bimolecular membrane substrate according to claim 6, the method further comprising,
prior to covering with the sealing liquid,
forming the microwell in a substrate body in which a first layer, an electrode, and a second layer are laminated to obtain the substrate, wherein
in obtaining the substrate, a through-hole is formed in the first layer, and the microwell surrounded by the through-hole and the electrode exposed in the through-hole is formed.
